(19) United States
(12) Patent Application Publication
(10) Pub. No.: US 2020/0315790 A1
(43) Pub. Date: Oct. 8, 2020

(54) TRANSCATHETER VALVE PROSTHESIS WITH A SEALING COMPONENT

(71) Applicant: MEDTRONIC VASCULAR, INC., Santa Rosa, CA (US)

(72) Inventors: Michael KRIVORUCHKO, Forestville, CA (US); Tracey TIEN, Tustin, CA (US)

(21) Appl. No.: 16/838,712

(22) Filed: Apr. 2, 2020

Related U.S. Application Data
(60) Provisional application No. 62/829,872, filed on Apr. 5, 2019.

(51) Int. Cl. A61F 2/24

(52) U.S. Cl. CPC A61F 2/2418; A61F 2220/0075; A61F 2230/0065

(57) ABSTRACT

A transcatheter valve prosthesis includes a stent, a prosthetic valve component disposed within and secured to the stent, and a sealing component. The sealing component includes a donut-shaped component and at least one pulling suture extending between the donut-shaped component and the stent. The at least one pulling suture is coupled to the stent such that the pulling suture can slide relative to the stent when the stent radially expands from the compressed configuration to the expanded configuration. The sealing component has a delivery configuration in which the donut-shaped component is disposed proximal of a proximal end of the stent and a deployed configuration in which the donut-shaped component is disposed distal of the proximal end of the stent. The pulling suture is configured to longitudinally pull the donut-shaped component as the stent radially expands from the compressed configuration to the expanded configuration.

### CROSS-REFERENCE TO RELATED APPLICATIONS

This application claims the benefit of U.S. Provisional Patent Application Ser. No. 62/829,872, filed Apr. 5, 2019, which is hereby incorporated by reference in its entirety for all purposes.

### FIELD OF THE INVENTION

The present invention relates to transcatheter valve prostheses having a sealing component configured to prevent paravalvular leakage.

### BACKGROUND OF THE INVENTION

A human heart includes four heart valves that determine the pathway of blood flow through the heart: the mitral valve, the tricuspid valve, the aortic valve, and the pulmonary valve. The mitral and tricuspid valves are atrioventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Recently, flexible prosthetic valves supported by stent structures that can be delivered percutaneously using a catheter-based delivery system have been developed for heart and venous valve replacement. These prosthetic valves may include either self-expanding or balloon-expandable stent structures with valve leaflets attached to the interior of the stent structure. The prosthetic valve can be reduced in diameter, by crimping onto a balloon catheter or by being contained within a sheath component of a delivery catheter, and advanced through the venous or arterial vasculature. Once the prosthetic valve is positioned at the treatment site, for instance within an incompetent native valve, the stent structure may be expanded to hold the prosthetic valve firmly in place.

Although transcatheter delivery methods have provided safer and less invasive methods for replacing a defective native heart valve, leakage between the implanted prosthetic valve and the surrounding native tissue is a recurring problem. Leakage sometimes occurs due to the fact that minimally invasive and percutaneous replacement of cardiac valves typically does not involve actual physical removal of the diseased or injured heart valve. Rather, the replacement stented prosthetic valve is delivered in a compressed condition to the valve site, where it is expanded to its operational state within the mitral valve. Calcified or diseased native leaflets are pressed to the side walls of the native valve by the radial force of the stent frame of the stented prosthetic valve. These calcified leaflets do not allow complete conformance of the stent frame with the native valve and can be a source of paravalvular leakage (PVL). Significant pressure gradients across the valve cause blood to leak through the gaps between the implanted stented prosthetic valve and the calcified anatomy.

Embodiments hereof are related to sealing components to seal gaps between the replacement stented prosthetic valve and native valve tissue.

### BRIEF SUMMARY OF THE INVENTION

Embodiments hereof relate to a transcatheter valve prosthesis that includes a stent, a prosthetic valve component disposed within and secured to the stent, and a sealing component. The stent has a compressed configuration for delivery within a vasculature and an expanded configuration for deployment within a native heart valve. The sealing component includes a donut-shaped component and at least one pulling suture extending between the donut-shaped component and the stent. The at least one pulling suture is coupled to the stent such that the pulling suture can slide relative to the stent when the stent radially expands from the compressed configuration to the expanded configuration. The sealing component has a delivery configuration in which the donut-shaped component is disposed proximal of a proximal end of the stent and a deployed configuration in which the donut-shaped component is disposed distal of the proximal end of the stent. The pulling suture is configured to longitudinally pull the donut-shaped component as the stent radially expands from the compressed configuration to the expanded configuration.

Embodiments hereof also relate to a transcatheter valve prosthesis includes a stent, a prosthetic valve component disposed within and secured to the stent, a sealing component, and at least one pulling suture extending between the sealing component and the stent. The stent has a compressed configuration for delivery within a vasculature and an expanded configuration for deployment within a native heart valve. A first end of the at least one pulling suture is attached to the stent, a second end of the at least one pulling suture is attached to the sealing component, and a body portion of the at least one pulling suture is coupled to the stent such that the pulling suture can slide relative to the stent when the stent radially expands from the compressed configuration to the expanded configuration. The sealing component has a delivery configuration in which the sealing component is disposed proximal of a proximal end of the stent and is longitudinally spaced apart from and not coupled to the stent except via the at least one pulling suture. The sealing component has a deployed configuration in which the sealing component is disposed distal of the proximal end of the stent and encircles an outer surface of the stent. The pulling suture is configured to longitudinally reposition the sealing component as the stent radially expands from the compressed configuration to the expanded configuration.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a transcatheter valve prosthesis including a sealing component according to an embodiment hereof, wherein a stent of the transcatheter valve prosthesis is in an expanded configuration and the sealing component is in a deployed configuration.

FIG. 2 is a schematic sectional view of the transcatheter valve prosthesis of FIG. 1 taken along line A-A of FIG. 1, wherein the stent of the transcatheter valve prosthesis is in a compressed configuration and the sealing component is in a delivery configuration.

FIG. 3 is a schematic sectional view of the transcatheter valve prosthesis of FIG. 1 taken along line A-A of FIG. 1, wherein the stent of the transcatheter valve prosthesis is in the expanded configuration and the sealing component is in the deployed configuration.

FIG. 4 is a perspective view of a portion of the transcatheter valve prosthesis of FIG. 1, wherein the stent of the transcatheter valve prosthesis is in the compressed configuration and the sealing component is in the delivery configuration, the sealing component including a plurality of pulling sutures and a cylindrical sleeve of the sealing component having a double pleat in the delivery configuration.

FIG. 5 is a perspective view of the portion of the transcatheter valve prosthesis of FIG. 4, wherein the stent of the transcatheter valve prosthesis is in the expanded configuration and the sealing component is in the deployed configuration and wherein the pulling sutures longitudinally pull the sealing component as the transcatheter valve prosthesis radially expands from a compressed configuration to an expanded configuration.

FIG. 6 is a side view of the transcatheter valve prosthesis of FIG. 1 illustrating a method step of assembling the sealing component onto the transcatheter valve prosthesis of FIG. 1.

FIG. 7 is a side view of the transcatheter valve prosthesis of FIG. 1 illustrating another method step of assembling the sealing component onto the transcatheter valve prosthesis of FIG. 1.

FIG. 8 is a schematic cross-sectional view of the transcatheter valve prosthesis of FIG. 1 illustrating another method step of assembling the sealing component onto the transcatheter valve prosthesis of FIG. 1, wherein a first end of each pulling suture is attached to the transcatheter valve prosthesis, a second end of each pulling suture is attached to an intermediate portion of the cylindrical sleeve, and a body portion of each pulling suture is coupled to the transcatheter valve prosthesis such that the pulling sutures can slide relative to the transcatheter valve prosthesis.

FIG. 9 is a schematic cross-sectional view of the transcatheter valve prosthesis of FIG. 1 taken along line B-B of FIG. 1, wherein the transcatheter valve prosthesis includes four pulling sutures.

FIG. 10 is a schematic cross-sectional view of a transcatheter valve prosthesis including a sealing component according to another embodiment hereof, wherein the transcatheter valve prosthesis includes three pulling sutures.

FIG. 11 is a schematic cross-sectional view of a transcatheter valve prosthesis including a sealing component according to another embodiment hereof, wherein the transcatheter valve prosthesis includes six pulling sutures.

FIG. 12 is a perspective view of a portion of a transcatheter valve prosthesis including a sealing component according to another embodiment hereof, wherein a stent of the transcatheter valve prosthesis is in a compressed configuration and the sealing component is in a delivery configuration and the sealing component further includes a plurality of restraining sutures.

FIG. 13 is a perspective view of the portion of the transcatheter valve prosthesis of FIG. 12, wherein the stent of the transcatheter valve prosthesis is in an expanded configuration and the sealing component is in a deployed configuration and the restraining sutures restrain the sealing component from extending radially outwards as the sealing component transforms from the delivery configuration to the deployed configuration.

FIG. 14 is a perspective view of a portion of a transcatheter valve prosthesis including a sealing component according to another embodiment hereof, wherein a stent of the transcatheter valve prosthesis is in a compressed configuration and the sealing component is in a delivery configuration, the sealing component including a plurality of pulling sutures and a cylindrical sleeve of the sealing component having a single pleat in the delivery configuration.

FIG. 15 is a perspective view of the portion of the transcatheter valve prosthesis of FIG. 14, wherein the stent of the transcatheter valve prosthesis is in an expanded configuration and the sealing component is in a deployed configuration and the pulling sutures longitudinally pull the sealing component as the transcatheter valve prosthesis radially expands from the compressed configuration to the expanded configuration.

FIG. 16 is a perspective view of a portion of a transcatheter valve prosthesis including a sealing component according to another embodiment hereof, wherein a stent of the transcatheter valve prosthesis is in a compressed configuration and the sealing component is in a delivery configuration, the sealing component including a plurality of pulling sutures and a cylindrical sleeve of the sealing component having no pleats in the delivery configuration.

FIG. 17 is a perspective view of the portion of the transcatheter valve prosthesis of FIG. 16, wherein the stent of the transcatheter valve prosthesis is in an expanded configuration and the sealing component is in a deployed configuration and the pulling sutures longitudinally pull the sealing component as the transcatheter valve prosthesis radially expands from the compressed configuration to the expanded configuration.

FIG. 18 is a perspective view of a portion of a transcatheter valve prosthesis including a sealing component according to another embodiment hereof, wherein a stent of the transcatheter valve prosthesis is in a compressed configuration and the sealing component is in a delivery configuration, the sealing component including a plurality of pulling sutures and a donut-shaped component.

FIG. 19 is a perspective view of the portion of the transcatheter valve prosthesis of FIG. 18, wherein the stent of the transcatheter valve prosthesis is in an expanded configuration and the sealing component is in a deployed configuration and the pulling sutures longitudinally pull the sealing component as the transcatheter valve prosthesis radially expands from the compressed configuration to the expanded configuration.

FIG. 20 is a perspective view of a portion of a transcatheter valve prosthesis including a sealing component according to another embodiment hereof, wherein a stent of the transcatheter valve prosthesis is in a compressed configuration and the sealing component is in a delivery configuration, the sealing component including a plurality of pulling sutures and a donut-shaped component, wherein the donut-shaped component is an undulating ring in a deployed configuration.

FIG. 21 is a perspective view of the portion of the transcatheter valve prosthesis of FIG. 20, wherein the stent of the transcatheter valve prosthesis is in an expanded configuration and the sealing component is in a deployed configuration and the pulling sutures longitudinally pull the sealing component as the transcatheter valve prosthesis radially expands from the compressed configuration to the expanded configuration.

FIG. 22 is a perspective view of the donut-shaped component of FIG. 21 in the deployed configuration, wherein the donut-shaped component is removed from the transcatheter vale prosthesis for illustrative purposes only.

FIG. 23 is a perspective view of a donut-shaped component in a deployed configuration according to another embodiment, wherein the donut-shaped component is removed from the transcatheter vale prosthesis for illustrative purposes only.

### DETAILED DESCRIPTION OF THE INVENTION

Specific embodiments of the present invention are now described with reference to the figures, wherein like reference numbers indicate identical or functionally similar elements. The terms “distal” and “proximal” in the following description refer to a position or direction relative to the direct of blood flow when a prosthesis is deployed at a treatment site. “Distal” and “distally” refer to positions in the direction of blood flow or downstream. “Proximal” and “proximally” refer to positions opposite the direction of blood flow or upstream. The following detailed description is merely exemplary in nature and is not intended to limit the invention or the application and uses of the invention. Although the description of the invention is in the context of treatment of heart valves, the invention may also be used where it is deemed useful in other valved intraluminal sites that are not in the heart. For example, the present invention may be applied to venous valves as well. Furthermore, there is no intention to be bound by any expressed or implied theory presented in the preceding technical field, background, brief summary or the following

### DETAILED DESCRIPTION

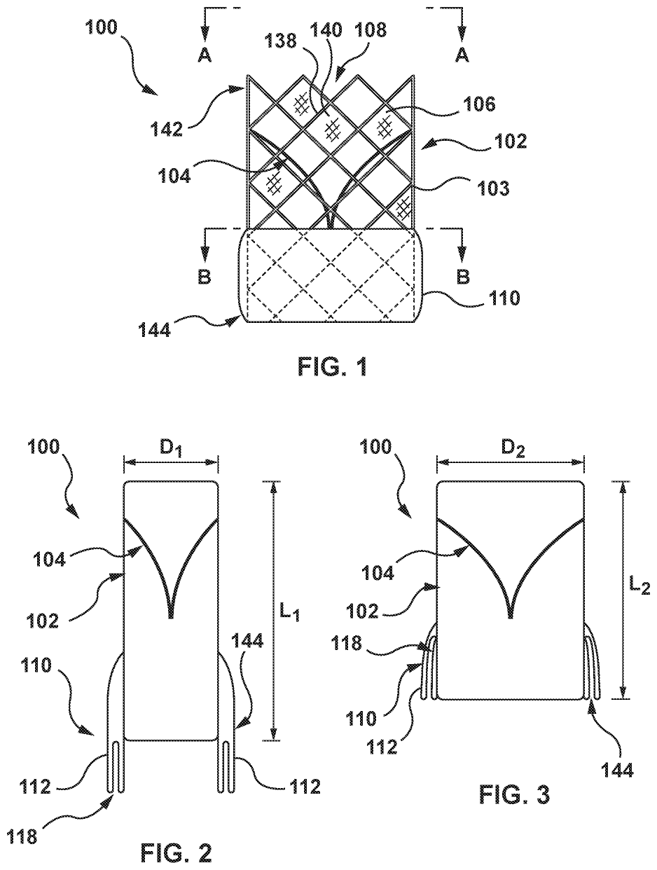

Embodiments hereof relate to a transcatheter valve prosthesis 100 having a radially-expandable stent 102 and a sealing component 110 that encircles or surrounds an outer surface 103 of the stent 102 in order to occlude or fill gaps between the perimeter of the transcatheter valve prosthesis 100 and the native valve annulus, thereby reducing, minimizing, or eliminating leaks there-between. FIG. 1 is a side view of the transcatheter valve prosthesis 100 including the stent 102 shown in an expanded configuration and the sealing component 110 shown in a deployed configuration. When the transcatheter valve prosthesis 100 is deployed within the valve annulus of a native heart valve, the stent 102 of the transcatheter valve prosthesis 100 is configured to be radially expanded within native valve leaflets of the patient's defective valve, to thereby retain the native valve leaflets in a permanently open state. The native valve annulus may include surface irregularities on the inner surface thereof, and as a result one or more gaps or cavities/crevices may be present or may form between the perimeter of transcatheter valve prosthesis 100 and the native valve annulus. For example, calcium deposits may be present on the native valve leaflets (e.g., stenotic valve leaflets) and/or shape differences may be present between the native heart valve annulus and the transcatheter valve prosthesis 100. More particularly, in some cases native annuli are not perfectly rounded and have indentations corresponding to the commissural points of the native valve leaflets. As a result, a prosthesis having an approximately circular cross-section does not provide an exact fit in a native valve. These surface irregularities, whatever their underlying cause, can make it difficult for conventional prosthetic valves to form a blood tight seal between the prosthetic valve and the inner surface of the valve annulus, causing undesirable paravalvular leakage and/or regurgitation at the implantation site. As will be described in more detail herein, the sealing component 110 is coupled to and encircles an outer circumferential surface of the stent 102 for sealing and preventing paravalvular leakage. The sealing component 110 functions to block any retrograde flow within the native valve, thereby preventing undesired regurgitation and preventing blood stagnation in and around the native valve sinuses. In addition, when the transcatheter valve prosthesis 100 is deployed, the sealing component 110 fills any/all gaps or cavities/crevices between the outer surface 103 of the stent 102 and native valve tissue such that blood flow through the target gap or cavity is occluded or blocked, or stated another way blood is not permitted to flow there-through. The sealing component 110 functions as a continuous circumferential seal around the transcatheter valve prosthesis 100 to block or prevent blood flow around the outer perimeter of the prosthesis, thereby minimizing and/or eliminating any paravalvular leakage at the implantation site.

The stent 102 of the transcatheter valve prosthesis 100 is a frame or scaffold that supports a prosthetic valve 104 including one or more valve leaflets within the interior of the stent 102. The prosthetic valve 104 is capable of blocking flow in one direction to regulate flow there-through via valve leaflets that may form a bicuspid or tricuspid replacement valve. The valve leaflets may be attached to a graft material 106 which encloses or lines a portion of the stent 102 as would be known to one of ordinary skill in the art of prosthetic tissue valve construction. The valve leaflets may be sutured or otherwise securely and sealingly attached along their bases to the interior surface of the graft material 106 or otherwise secured to the stent 102. Adjoining pairs of leaflets are attached to one another at their lateral ends to form commissures. If the transcatheter valve prosthesis 100 is configured for placement within a native valve having three leaflets such as the aortic, tricuspid, or pulmonary valves, the prosthetic valve component 104 may include three valve leaflets. If transcatheter valve prosthesis 100 is configured for placement within a native valve having two leaflets such as the mitral valve, the prosthetic valve component 104 may include two valve leaflets.

Leaflets may be made of pericardial material; however, the leaflets may instead be made of another material. Natural tissue for replacement valve leaflets may be obtained from, for example, heart valves, aortic roots, aortic walls, aortic leaflets, pericardial tissue, such as pericardial patches, bypass grafts, blood vessels, intestinal submucosal tissue, umbilical tissue and the like from humans or animals. Synthetic materials suitable for use as valve leaflets include DACRON® polyester commercially available from Invista North America S.A.R.L. of Wilmington, Del., other cloth materials, nylon blends, polymeric materials, and vacuum deposition nitinol fabricated materials. One polymeric material from which the leaflets can be made is an ultra-high molecular weight polyethylene material commercially available under the trade designation DYNEEMA from Royal DSM of the Netherlands. With certain leaflet materials, it may be desirable to coat one or both sides of the leaflet with a material that will prevent or minimize overgrowth. It is further desirable that the leaflet material is durable and not subject to stretching, deforming, or fatigue.

The stent 102 will now be described in more detail. The stent 102 is illustrated herein in order to facilitate description of the sealing component 110 attached thereto for preventing and/or repairing paravalvular leakage according to embodiments hereof. It is understood that any number of alternate stents or frames can be used with the sealing components for preventing and/or repairing paravalvular leakage described herein. The configuration of the stent 102 is merely exemplary.

In the embodiment of FIG. 1, the stent 102 is a patterned tubular component that is radially expandable from a compressed or delivery configuration to an expanded or deployed configuration as described in more detail herein. The stent 102 may be a unitary, generally tubular component defining a central lumen or passageway 108 and having a first or distal end 142 and a second or proximal end 144. The transcatheter valve prosthesis 100 is configured for replacement for an aortic valve such that the proximal end 144 functions as an inflow end of the transcatheter valve prosthesis 100 and extends into and anchors within the aortic annulus of a patient's left ventricle, while the distal end 142 functions as an outflow end of the transcatheter valve prosthesis 100 and is positioned in the patient's ascending aorta or aortic sinuses. The stent 102 includes a plurality of struts 138 that form a plurality of side openings 140. Stated another way, the plurality of struts 138 define the plurality of side openings 140 of the stent 102. In an embodiment, the plurality of side openings 140 may be diamond-shaped. The stent 102 may be formed by a laser-cut manufacturing method and/or another conventional stent/scaffold forming method, as would be understood by one of ordinary skill in the art. However, it will be understood by one of ordinary skill in the art that the illustrated configuration of the stent 102 is exemplary and the stent 102 may have an alternative pattern or configuration. For example, as alternatives to the expanded configuration of FIG. 1, the stent 102 may have an expanded or deployed configuration in which the distal end 142 is enlarged or flared relative to the proximal end 144. In another embodiment, the stent 102 may have an hourglass configuration or profile, or other stent configurations or shapes known in the art for valve replacement. The cross-section of the stent 102 may be circular, ellipsoidal, rectangular, hexagonal, square, or other polygonal shape, although at present it is believed that circular or ellipsoidal may be preferable when the transcatheter valve prosthesis 100 is being provided for replacement of the mitral or aortic valves.

Graft material 106 may enclose or line the stent 102 as would be known to one of ordinary skill in the art of prosthetic tissue valve construction. Graft material 106 may be a natural or biological material such as pericardium or another membranous tissue such as intestinal submucosa. Alternatively, graft material 106 may be a low-porosity woven fabric, such as polyester, Dacron fabric, or PTFE, which creates a one-way fluid passage when attached to the stent. In one embodiment, graft material 106 may be a knit or woven polyester, such as a polyester or PTFE knit, which can be utilized when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example.

In embodiments hereof, the stent 102 is self-expanding to return to an expanded or deployed state or configuration from a compressed or constricted delivery state or configuration and may be made from stainless steel, a pseudo-elastic metal such as a nickel titanium alloy or Nitinol, or a so-called super alloy, which may have a base metal of nickel, cobalt, chromium, or other metal. “Self-expanding” as used herein means that a structure/component has a mechanical memory to return to the expanded or deployed configuration. Mechanical memory may be imparted to the wire or structure that forms the stent 102 by thermal treatment to achieve a spring temper in stainless steel, for example, or to set a shape memory in a susceptible metal alloy, such as nitinol, or a polymer, such as any of the polymers disclosed in U.S. Pat. Appl. Pub. No. 2004/0111111 to Lin, which is incorporated by reference herein in its entirety.

Alternatively, the stent 102 may be balloon-expandable as would be understood by one of ordinary skill in the art. If balloon-expandable, the stent 102 is made from a plastically deformable material such that when expanded by a dilatation balloon, the stent 102 maintains its radially expanded configuration. The stent 102 may be formed from stainless steel or other suitable metals, such as platinum iridium, cobalt chromium alloys such as MP35N, or various types of polymers or other materials known to those skilled in the art, including said materials coated with various surface deposits to improve clinical functionality.

Delivery of the transcatheter valve prosthesis 100 may be accomplished via a percutaneous transfemoral approach or a transapical approach directly through the apex of the heart via a thoracotomy, or may be positioned within the desired area of the heart via different delivery methods known in the art for accessing heart valves. During delivery, if self-expanding, the transcatheter valve prosthesis 100 remains compressed until it reaches a target diseased native heart valve, at which time the transcatheter valve prosthesis 100 can be released from the delivery catheter and permitted to expand in situ via self-expansion. The delivery catheter is then removed and the transcatheter valve prosthesis 100 remains deployed within the native target heart valve. Alternatively, as described above, the transcatheter valve prosthesis 100 may be balloon-expandable and delivery thereof may be accomplished via a balloon catheter as would be understood by one of ordinary skill in the art.

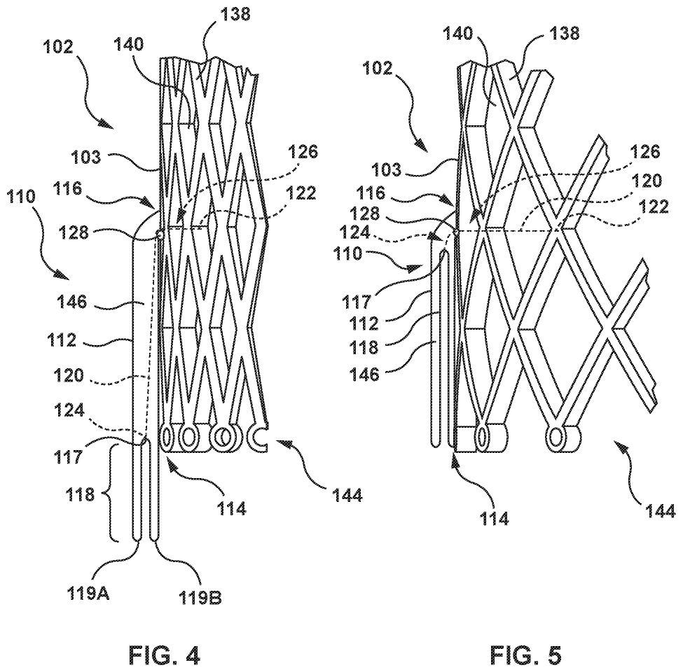

The sealing component 110 will now be described in more detail with reference to FIGS. 2-5. FIG. 2 is a schematic sectional view of the transcatheter valve prosthesis 100 taken along line A-A of FIG. 1 with the stent 102 being shown in a compressed configuration and the sealing component 110 being shown in a delivery configuration, while FIG. 3 is a schematic sectional view of the transcatheter valve prosthesis 100 taken along line A-A of FIG. 1 with the stent 102 shown in the expanded configuration and the sealing component 110 is in the deployed configuration. The sealing component 110 includes a cylindrical sleeve 112 formed of a flexible material and a plurality of pulling sutures 120 (shown in FIGS. 4 and 5) extending between the cylindrical sleeve 112 and the stent 102. The plurality of pulling sutures 120 are coupled to the stent 102 such that each pulling suture 120 can slide relative to the stent 102 when the stent 102 radially expands from the compressed configuration to the expanded configuration. Suitable flexible materials for the cylindrical sleeve 112 include but are not limited to a low-porosity woven fabric, such as polyester, Dacron fabric, or PTFE. Porous materials advantageously provide a medium for tissue ingrowth. Further, the cylindrical sleeve 112 may be pericardial tissue or may be a knit or woven polyester, such as a polyester or PTFE knit, both of which provide a medium for tissue ingrowth and have the ability to stretch to conform to a curved surface. Polyester velour fabrics may alternatively be used, such as when it is desired to provide a medium for tissue ingrowth on one side and a smooth surface on the other side.

In operation, radial expansion of the stent 102 from the compressed configuration to the expanded configuration causes the pulling suture 120 to longitudinally reposition an intermediate portion 118 of the cylindrical sleeve 112. More particularly, when the stent 102 is in the compressed configuration, the stent 102 has a first length L₁ and a first diameter D₁. When the stent 102 radially expands into the expanded configuration, the stent 102 has a second length L₂ which is less than the first length L₁ and a second diameter D₂ which is greater than the first diameter D₁. In embodiments hereof, expansion of the stent 102 is utilized to reposition the cylindrical sleeve 112 as the transcatheter valve prosthesis 100 radially expands. More particularly, when the stent 102 is in the compressed configuration, the intermediate portion 118 of the cylindrical sleeve 112 is disposed at a first longitudinal position proximal of the proximal end 144 of the stent 102. The pulling sutures 120 longitudinally pull, draw, haul, drag, move or otherwise reposition the intermediate portion 118 of the cylindrical sleeve 112 as the transcatheter valve prosthesis 100 radially expands from the compressed configuration to the expanded configuration. Thus, when the stent 102 is in the expanded configuration, the intermediate portion 118 of the cylindrical sleeve 112 is disposed at a second longitudinal portion that is distal of the proximal end 144 of the stent 102.

Advantageously, a large majority of the flexible material of the sealing component 110 is disposed proximal to the proximal end 144 of the stent 102 when the transcatheter valve prosthesis 100 is disposed within a delivery system for delivery thereof. Accordingly, the addition of the sealing component 110 advantageously does not increase, or minimally increases, the packing profile of transcatheter valve prosthesis 100 so that transcatheter valve prosthesis 100 has the ability to pack in lower profile delivery systems. When transcatheter valve prosthesis 100 is deployed, as shown in FIG. 3, the stent 102 radially expands and the pulling sutures 120 pull or move the sealing component 110 into position such that the intermediate portion 118 of the sealing component 110 is now disposed distal to the proximal end 144 of the stent 102. Stated another way, the transcatheter valve prosthesis 100 is configured such that the sealing component 110 is primarily disposed beyond the proximal end 144 of the stent 102 during delivery and then the sealing component 110 is pulled and bunched up adjacent to the inflow region of the transcatheter valve prosthesis 100 during stent deployment so that the sealing component 110 is now in position to prevent and/or repair paravalvular leakage. The deployment and repositioning of the sealing component 110 thus does not require user intervention but rather utilizes the expansion of the stent 102 to deploy and reposition the sealing component 110.

The components and deployment of the sealing component 110 are shown in greater detail in FIGS. 4 and 5. FIG. 4 is a perspective view of a portion of the transcatheter valve prosthesis 100 with the stent 102 being shown in the compressed configuration and the sealing component 110 being shown in the delivery configuration, while FIG. 5 is a perspective view of a portion of the transcatheter valve prosthesis 100 with the stent 102 being shown in the expanded configuration and the sealing component 110 being shown in the deployed configuration. The cylindrical sleeve 112 is a cylindrical or component defining a central lumen or passageway and having a first circumferential edge 114 and an opposing or second circumferential edge 116. As best shown in FIGS. 4 and 5, the first circumferential edge 114 of the cylindrical sleeve 112 is attached to the outer surface 103 of the stent 102 around the proximal end 144 of the stent 102 and the second circumferential edge 116 of the cylindrical sleeve 112 is attached to the outer surface 103 of the stent 102 distal of the proximal end 144 of the stent 102. The first and second circumferential edges 114, 116 of the cylindrical skirt 112 may be attached to the stent 102 by any suitable means known to those skilled in the art, for example and not by way of limitation, suture/stitches, welding, adhesive, or mechanical coupling.

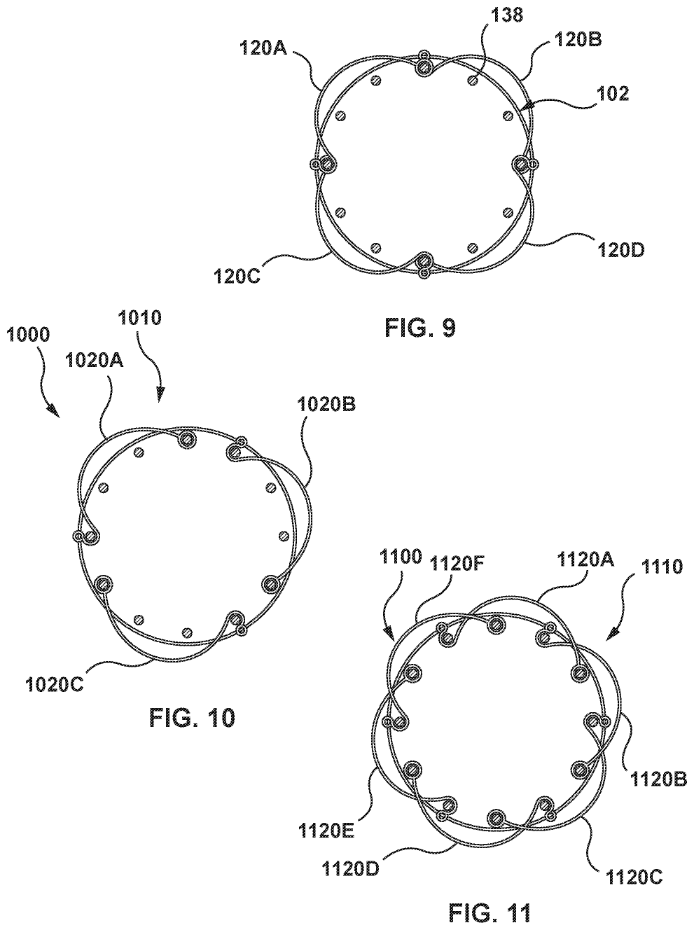

In FIGS. 4 and 5, the pulling suture 120 is shown in phantom to readily distinguish the pulling suture 120 from the cylindrical sleeve 112. Since FIGS. 4 and 5 illustrate only a portion of the transcatheter valve prosthesis 100, only a single pulling suture 120 is shown. However, in the embodiment of FIGS. 1-5, the sealing component 110 includes a total of four pulling sutures. More particularly, as best shown in FIG. 9 which is a schematic cross-sectional view of the transcatheter valve prosthesis 100 taken along line B-B of FIG. 1, four pulling sutures 120A, 120B, 120C, 120D are equally circumferentially spaced around the stent 102 so that the pulling sutures 120A, 120B, 120C, 120D are configured to uniformly pull or draw up the cylindrical sleeve 112. The pulling sutures 120A, 120B, 120C, 120D are collectively or individually referred to herein as the pulling sutures 120 or the pulling suture 120, respectively.

Each pulling suture 120 is an elongated component having a first end 122 and a second or opposing end 124 with a body portion 126 extending between the first and second ends 122, 124. The first end 122 of the pulling suture 120 is fixedly attached to the stent 102, the second end 124 of the pulling suture 120 is fixedly attached to the intermediate portion 118 of the cylindrical sleeve 112, and the body portion 126 of the pulling suture 120 is coupled to the stent 102 such that the pulling suture 120 can slide relative to the stent 102. The slidability of the pulling suture 120 will be described in more detail with reference to FIG. 8 described in more detail herein.

Although described with a total of four pulling sutures, it will be understood by those of ordinary skill in the art that the sealing component 110 may include more or fewer pulling sutures for repositioning the intermediate portion 118 of the cylindrical sleeve 112. For example, FIG. 10 is a schematic cross-sectional view of a transcatheter valve prosthesis 1000 including a sealing component 1010 according to another embodiment hereof in which the sealing component 1010 includes a total of three pulling sutures 1020A, 1020B, 1020C equally circumferentially spaced around the transcatheter valve prosthesis 1000. As another example, FIG. 11 is a schematic cross-sectional view of a transcatheter valve prosthesis 1100 including a sealing component 1110 according to another embodiment hereof in which the sealing component 1110 includes a total of six pulling sutures 1120A, 1120B, 1120C, 1120D, 1120E, 1120F equally circumferentially spaced around the transcatheter valve prosthesis 1100. It will be understood by those of ordinary skill in the art that at least one pulling suture is necessary to reposition the intermediate portion 118 of the cylindrical sleeve 112.

In the embodiment of FIGS. 1-5, the intermediate portion 118 of the cylindrical sleeve 112 includes a double pleat in the delivery configuration of the sealing component 110. Stated another way, the intermediate portion 118 includes a first fold or pleat 119A formed thereon and a second fold or pleat 119B formed thereon. The first and second folds or pleats 119A, 119B are formed on the cylindrical sleeve 112 as a result of the manner in which the cylindrical sleeve 112 is attached to the stent 102 and the pulling suture(s) 120. The first and second folds or pleats are disposed side by side, or directly adjacent to one another, with a crest 117 disposed therebetween. As shown in FIGS. 2 and 4, in the delivery configuration of the sealing component 110, the intermediate portion 118 including both first and second folds or pleats 119A, 119B is disposed proximal of the proximal end 144 of the stent 102. The second end 124 of the pulling suture 120 is attached to the crest 117 disposed between the first and second folds or pleats 119A, 119B of the intermediate portion 118 of the cylindrical sleeve 112. In an embodiment, the crest 117 is longitudinally aligned with the proximal end 144 of the stent 102 when the sealing component 110 is in the delivery configuration.

As described above, the pulling suture 120 is configured to longitudinally pull, draw, haul, drag, move or otherwise reposition the intermediate portion 118 of the cylindrical sleeve 112 as the stent 102 radially expands from the compressed configuration to the expanded configuration. More particularly, the body portion 126 of the pulling suture 120A is slidingly disposed through a hole or opening 128 formed within a strut 138 of the stent 102. Alternatively, the body portion 126 of the pulling suture 120A may be woven through one or more side openings 140 of the stent 102 such that the body portion 1226 of the pulling suture 120 can slide relative to the stent 102 when the stent 102 radially expands from the compressed configuration to the expanded configuration. When the stent 102 radially expands, the intermediate portion 118 of the cylindrical sleeve 112 folds or bunches into the space or gap 146 defined between the outer surface 103 of the stent 102 and the inner surface of the cylindrical sleeve 112. Stated another way, the intermediate portion 118 of the cylindrical sleeve 112 is pulled in a distal direction, i.e., towards the distal end 142 of the stent 102, by the radial expansion of the stent 102 as the struts 138 separate and the side openings 140 widen during the stent expansion process. In particular, because the first end 122 of the pulling suture 120A is fixedly attached to one of the struts 138 of the stent 102, the first end 122 moves with the strut 138. When the stent 102 radially expands, the distance between the first end 122 of the pulling suture 120A and the hole 128 (formed in an adjacent strut 138) increases. Thus, a longer length of the body portion 126 of the pulling suture 120A is taken between the strut 138 and the hole 128. Thus, a smaller length of the body portion 126 of the pulling suture 120A remains between the hole 128 and the second end 124 of the pulling suture 120A attached to the intermediate portion 118 of the cylindrical sleeve 112, thereby pulling the intermediate portion 118 closer to the hole 128. In the deployed configuration of the sealing component 110 as best shown in FIG. 5, the intermediate portion 118 of the cylindrical sleeve 110 is disposed distal of the proximal end 144 of the stent 102 and encircles the outer surface 103 of the stent 102. In this embodiment, the entire cylindrical sleeve 112 is disposed distal of the proximal end 144 of the stent 102 in the deployed configuration of the sealing component 110. Further, in this embodiment, the sealing component 110 in the deployed configuration includes three overlapping layers of the flexible material of the cylindrical sleeve 112.

When deployed in situ, the sealing component 110 may be positioned in situ at the native valve annulus, slightly above the valve annulus, slightly below the valve annulus, or some combination thereof. Longitudinal placement and/or the size and shape of the sealing component 110 may be adjusted or adapted according to each application and to a patient's unique needs. For example, depending on the anatomy of the particular patient, the positioning of the second circumferential edge 116 of the cylindrical sleeve 112 relative to the stent 102 may be selected or adjusted so that in situ the sealing component 110 is positioned between the transcatheter valve prosthesis 100 and the interior surfaces of the native valve leaflets, between the transcatheter valve prosthesis 100 and the interior surfaces of the native valve annulus, and/or between the transcatheter valve prosthesis 100 and the interior surfaces of the left ventricular outflow track (LVOT).

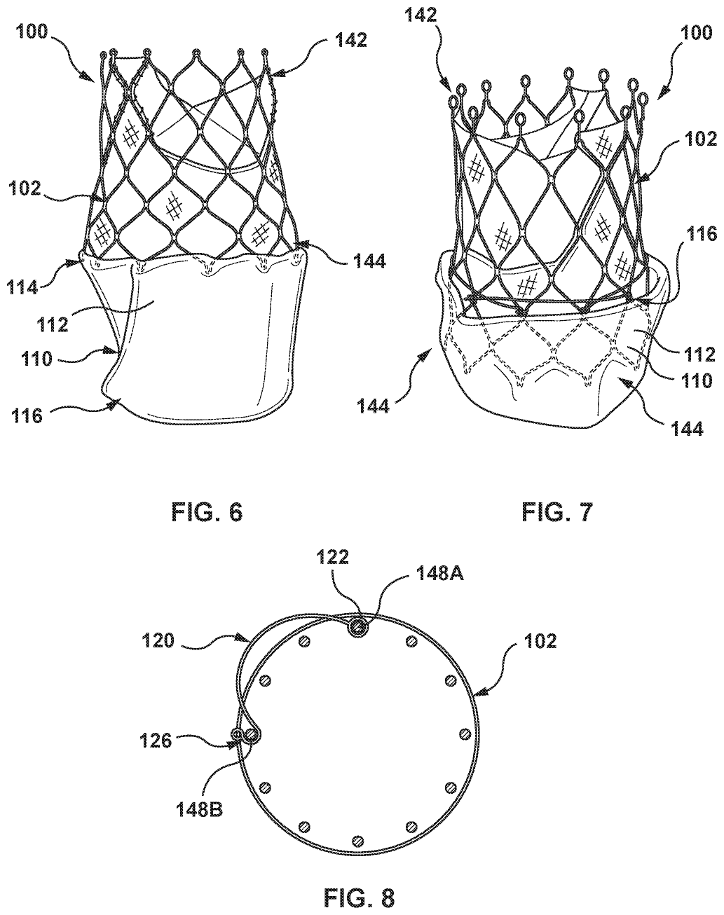

FIGS. 6, 7, and 8 illustrate method steps of assembling the sealing component 110 onto the transcatheter valve prosthesis 100. In FIG. 6, the first circumferential edge 114 of the cylindrical sleeve 112 is disposed around and attached to the proximal end 144 of the stent 102. The first circumferential edge 114 of the cylindrical sleeve 112 may be attached to the proximal end 144 of the stent 102 along the entire perimeter thereof with a running stitch. The second circumferential edge 116 of the cylindrical sleeve 112 may also be seamed with a running stitch but is not yet attached to the stent 102. Next, as shown in FIG. 7, the cylindrical sleeve 112 is folded or inverted like a sock so that the second circumferential edge 116 of the cylindrical sleeve 112 is disposed adjacent to the stent 102, distal to the proximal end 144 thereof. In an embodiment, approximately one-third (⅓) of the total length of the cylindrical sleeve 112 extends beyond the proximal end 144 of the stent 102. The second circumferential edge 116 of the cylindrical sleeve 112 is disposed adjacent to the stent 102 such that the cylindrical sleeve 112 covers the first set of diamond-shaped side openings 140 adjacent to the proximal end 144 of the stent 102.

Once the cylindrical sleeve 112 is inverted as desired, the pulling sutures 120 are put into place. FIG. 8 is a schematic cross-sectional view of the transcatheter valve prosthesis 100 showing placement of only a single pulling suture 120 for clarity. Although FIG. 8 illustrates positioning of only a single pulling suture, the other three pulling sutures are similarly attached to the stent 102. The first end 122 of the pulling suture 120A is fixedly attached to the stent 102 at a first node 148A of the stent 102. The body portion 126 of the pulling suture 120A is threaded through the hole 128 positioned through a strut 138 at a second node 148B of the stent 102. The spacing between the first node 148A and the second node 148B of the stent 102 may vary according to application and the desired amount of repositioning of the intermediate portion 118 of the cylindrical sleeve 112. In an embodiment, the first node 148A and the second node 148B of the stent 102 are spaced apart by two other nodes of the stent 102 as shown in FIG. 8. The intermediate portion 118 of the cylindrical sleeve 112 may then be folded into the double pleat configuration, and the second end 124 of the pulling suture 120 (not shown in FIG. 8) is attached to the crest 117 disposed between the first and second folds or pleats 119A, 119B of the intermediate portion 118 of the cylindrical sleeve 112 as described above with reference to FIG. 4. After all of the pulling sutures 120 are in place, at least some portion of the second circumferential edge 116 of the cylindrical sleeve 112 is be attached to the stent 102 along the entire perimeter thereof with a running stitch. In an embodiment, the second circumferential edge 116 of the cylindrical sleeve 112 is attached to the stent 102 around the proximal end 144 thereof. However, in another embodiment, the second circumferential edge 116 of the cylindrical sleeve 112 is attached to the stent 102 anywhere along the axial length of the frame depending on the desired placement of the sealing component 110.

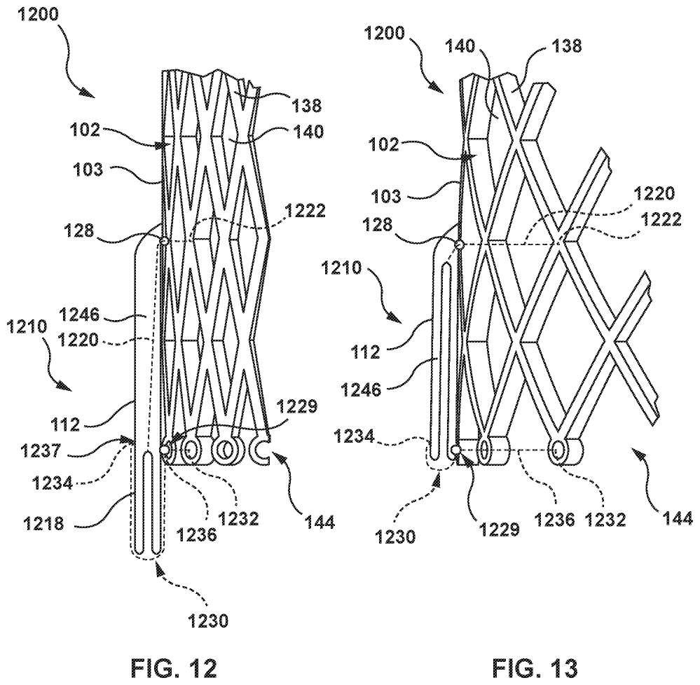

In another embodiment hereof, the transcatheter valve prosthesis may further include a plurality of restraining sutures. More particularly, FIG. 12 is a perspective view of a portion of a transcatheter valve prosthesis 1200 including the stent 102 shown in the compressed configuration and a sealing component 1210 shown in a delivery configuration while FIG. 13 is a perspective view of the portion of the transcatheter valve prosthesis 1200 with the stent 102 shown in the expanded configuration and the sealing component 1210 shown in a deployed configuration. The sealing component 1210 is similar to the sealing component 110. The sealing component 1210 includes a cylindrical sleeve 1212 formed of a flexible material and a plurality of pulling sutures 1220 extending between the cylindrical sleeve 1212 and the stent 102. The plurality of pulling sutures 1220 are coupled to the stent 102 such that a body portion 1226 of each pulling suture 1220 can slide relative to the stent 102 when the stent 102 radially expands from the compressed configuration to the expanded configuration. The cylindrical sleeve 1212 is a cylindrical or tubular component defining a central lumen or passageway and having a first circumferential edge 1214 and an opposing or second circumferential edge 1216. The first circumferential edge 1214 of the cylindrical sleeve 1212 is attached to the outer surface of the transcatheter valve prosthesis 1200 around a proximal end 144 of the stent 102 and the second circumferential edge 1216 of the cylindrical sleeve 1212 is attached to the outer surface of the transcatheter valve prosthesis 1200 distal of the proximal end 144 of the stent 102.

In FIGS. 12 and 13, the pulling suture 1220 is shown in phantom to readily distinguish the pulling suture 1220 from the cylindrical sleeve 1212. The pulling suture 1220 is an elongated component having a first end 1222 and a second or opposing end 1224 with a body portion 1226 extending between the first and second ends 1222, 1224. The first end 1222 of the pulling suture 1220 is fixedly attached to the stent 102, the second end 1224 of the pulling suture 1220 is fixedly attached to an intermediate portion 1218 of the cylindrical sleeve 1212, and the body portion 1226 of the pulling suture 1220 is coupled to the stent 102 such that the pulling suture 1220 can slide relative to the stent 102. Similar to the pulling suture 120, the pulling suture 1220 is configured to longitudinally pull, draw, haul, drag, move or otherwise reposition the intermediate portion 1218 of the cylindrical sleeve 1212 as the stent 102 radially expands from the compressed configuration to the expanded configuration. More particularly, the body portion 1226 of the pulling suture 1220A is slidingly disposed through the hole or opening 128 formed within a strut 138 of the stent 102. When the stent 102 radially expands, the intermediate portion 1218 of the cylindrical sleeve 1122 folds or bunches into the space or gap 1246 defined between the outer surface 103 of the stent 102 and the inner surface of the cylindrical sleeve 1212. In particular, because the first end 1222 of the pulling suture 1220A is fixedly attached to one of the struts 138 of the stent 102, the first end 1222 moves with the strut 138. When the stent 102 radially expands, the distance between the first end 1222 of the pulling suture 1220A and the hole 128 (formed in an adjacent strut 138) increases. Thus, a longer length of the body portion 1226 of the pulling suture 1220A is taken between the strut 138 and the hole 128. Thus, a smaller length of the body portion 1226 of the pulling suture 1220A remains between the hole 128 and the second end 1224 of the pulling suture 1220A attached to the intermediate portion 1218 of the cylindrical sleeve 1212, thereby pulling the intermediate portion 1218 closer to the hole 128.

In the deployed configuration of the sealing component 1210, the intermediate portion 1218 of the cylindrical sleeve 1210 is disposed distal of the proximal end 144 of the stent 102 and encircles the outer surface 103 of the stent 102. In this embodiment, the entire cylindrical sleeve 1212 is disposed distal of the proximal end 144 of the stent 102 in the deployed configuration of the sealing component 1210. Further in this embodiment, the sealing component 1210 in the deployed configuration includes three overlapping layers of the flexible material of the cylindrical sleeve 1212.

The sealing component 1210 further includes a plurality of restraining sutures 1230 extending between the cylindrical sleeve 1212 and the stent 102. Since FIGS. 12 and 13 illustrate only a portion of the transcatheter valve prosthesis 1200, only a single restraining suture 1230 is shown. However, in the embodiment of FIGS. 12 and 13, the sealing component 1210 includes a total of four restraining sutures 1230 that are equally circumferentially spaced around the stent 102. The plurality of restraining sutures 1230 are configured to restrain the sealing component 1210 from extending radially outwards as the sealing component 1210 transforms from the delivery configuration to the deployed configuration. In FIGS. 12 and 13, the restraining suture 1230 is shown in phantom to readily distinguish the restraining suture 1230 from the cylindrical sleeve 1212. Each restraining suture 1230 is longitudinally or axially spaced apart from each pulling suture 1220 but the sutures 1230, 1220 are circumferentially aligned.

The restraining suture 1230 is an elongated component having a first end 1232 and a second or opposing end 1234 with a body portion 1236 extending between the first and second ends 1232, 1234. The first end 1232 of the restraining suture 1230 is fixedly attached to the stent 102, the second end 1234 of the restraining suture 1230 is fixedly attached to a second intermediate portion 1237 of the cylindrical sleeve 1212, and the body portion 1236 of the restraining suture 1230 is coupled to the stent 102 such that the restraining suture 1230 can slide relative to the stent 102 when the stent 102 radially expands from the compressed configuration to the expanded configuration. More particularly, the body portion 1236 of the restraining suture 1230 is slidingly disposed through a hole or opening 1229 formed within a strut 138 of the stent 102. The restraining suture 1230 is configured to restrain, hold, or otherwise prevent the cylindrical sleeve 1212 from extending radially outwards as the sealing component 1210 transforms from the delivery configuration to the deployed configuration. In an embodiment, the second intermediate portion 1237 of the cylindrical sleeve 1212 is distal to the intermediate portion 1218 of the cylindrical sleeve 1212. The plurality of restraining sutures 1230 actuate simultaneously with the plurality of pulling sutures 1220 to ensure that the cylindrical sleeve 1212 does not flip radially outwards towards the distal end of the transcatheter valve prosthesis 1200. In particular, because the first end 1232 of the restraining suture 1230 is fixedly attached to one of the struts 138 of the stent 102, the first end 122 moves with the strut 138. When the stent 102 radially expands, the distance between the first end 1232 of the restraining suture 1230 and the hole 1229 (formed in an adjacent strut 138) increases. Thus, a longer length of the body portion 1236 of the restraining suture 1230 is taken between the strut 138 and the hole 1229. Thus, the length of the body portion 1236 of the restraining suture 1230 extending between the hole 1229 and the second end 1234 of the restraining suture 1230 attached to the second intermediate portion 1237 of the cylindrical sleeve 1212 decreases, thereby restraining or preventing the cylindrical sleeve 1212 from flipping radially outwards as the second intermediate portion 1237 is pulled closer towards the hole 1229.

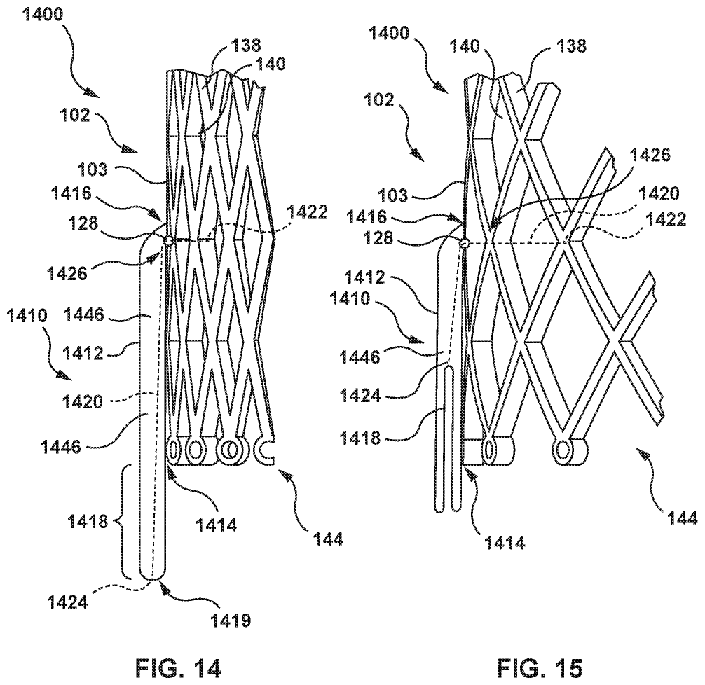

FIGS. 14 and 15 illustrate another embodiment of a sealing component 1410 in which the sealing component 1410 has a single pleat configuration in the delivery configuration rather than the double pleat configuration of the sealing component 110. More particularly, FIG. 14 is a perspective view of a portion of a transcatheter valve prosthesis 1400 including the stent 102 shown in the compressed configuration and the sealing component 1410 shown in a delivery configuration while FIG. 15 is a perspective view of the portion of the transcatheter valve prosthesis 1400 with the stent 102 shown in the expanded configuration and the sealing component 1410 shown in a deployed configuration. The sealing component 1410 is similar to the sealing component 110 except that the sealing component 1410 has a single pleat configuration in the delivery configuration rather than the double pleat configuration of the sealing component 110. The sealing component 1410 includes a cylindrical sleeve 1412 formed of a flexible material and a plurality of pulling sutures 1420 extending between the cylindrical sleeve 1412 and the stent 102. The plurality of pulling sutures 1420 are coupled to the stent 102 such that a body portion 1426 of each pulling suture 1420 can slide relative to the stent 102 when the stent 102 radially expands from the compressed configuration to the expanded configuration. The cylindrical sleeve 1412 is a cylindrical or component defining a central lumen or passageway and having a first circumferential edge 1414 and an opposing or second circumferential edge 1416. The first circumferential edge 1414 of the cylindrical sleeve 1412 is attached to the outer surface of the transcatheter valve prosthesis 1400 around the proximal end 144 of the stent 102 and the second circumferential edge 1416 of the cylindrical sleeve 1412 is attached to the outer surface of the transcatheter valve prosthesis 1400 distal of the proximal end 144 of the stent 102.

In FIGS. 14 and 15, the pulling suture 1420 is shown in phantom to readily distinguish the pulling suture 1420 from the cylindrical sleeve 1412. The pulling suture 1420 is an elongated component having a first end 1422 and a second or opposing end 1424 with a body portion 1426 extending between the first and second ends 1422, 1424. The first end 1422 of the pulling suture 1420 is attached to the stent 102, the second end 1424 of the pulling suture 1420 is attached to the intermediate portion 1418 of the cylindrical sleeve 1412, and the body portion 1426 of the pulling suture 1420 is coupled to the stent 102 such that the pulling suture 1420 can slide relative to the stent 102. In this embodiment, the intermediate portion 1418 of the cylindrical sleeve 1412 includes a single pleat in the delivery configuration. Stated another way, the intermediate portion 1418 includes a single fold or pleat 1419 formed thereon. In the delivery configuration of the sealing component 1410, the intermediate portion 1418 including the single fold or pleat 1419 is disposed proximal of the proximal end 144 of the stent 102. The second end 1424 of the pulling suture 1420 is attached to the single fold or pleat 1419 of the intermediate portion 1418 of the cylindrical sleeve 1412.

Similar to the pulling suture 120, the pulling suture 1420 is configured to longitudinally pull, draw, haul, drag, move or otherwise reposition the intermediate portion 1418 of the cylindrical sleeve 1412 as the stent 102 radially expands from the compressed configuration to the expanded configuration. More particularly, the body portion 1426 of the pulling suture 1420A is slidingly disposed through a hole or opening 128 formed within a strut 138 of the stent 102. When the stent 102 radially expands, the intermediate portion 1418 of the cylindrical sleeve 1412 folds or bunches into the space or gap 1446 defined between the outer surface 103 of the stent 102 and the inner surface of the cylindrical sleeve 1412. In the deployed configuration of the sealing component 1410, the intermediate portion 1418 of the cylindrical sleeve 1410 is disposed distal of the proximal end 144 of the stent 102 and the cylindrical sleeve 1412 encircles the outer surface 103 of the stent 102. In particular, because the first end 1422 of the pulling suture 1420A is fixedly attached to one of the struts 138 of the stent 102, the first end 1422 moves with the strut 138. When the stent 102 radially expands, the distance between the first end 1422 of the pulling suture 1420A and the hole 128 (formed in an adjacent strut 138) increases. Thus, a longer length of the body portion 1426 of the pulling suture 1420A is taken between the strut 138 and the hole 128. Thus, a smaller length of the body portion 1426 of the pulling suture 1420A remains between the hole 128 and the second end 1424 of the pulling suture 1420A attached to the intermediate portion 1418 of the cylindrical sleeve 1412, thereby pulling the intermediate portion 1418 closer to the hole 128.

In this embodiment, the sealing component 1410 in the deployed configuration includes three overlapping layers of the flexible material of the cylindrical sleeve 1412. Further, in this embodiment, the sealing component 1410 is disposed both proximal to and distal to the proximal end 144 of the stent 102 when the sealing component 1410 is in the deployed configuration. In another embodiment (now shown), the sealing component 1410 is disposed only distal to the proximal end 144 of the stent 102 when the sealing component 1410 is in the deployed configuration.

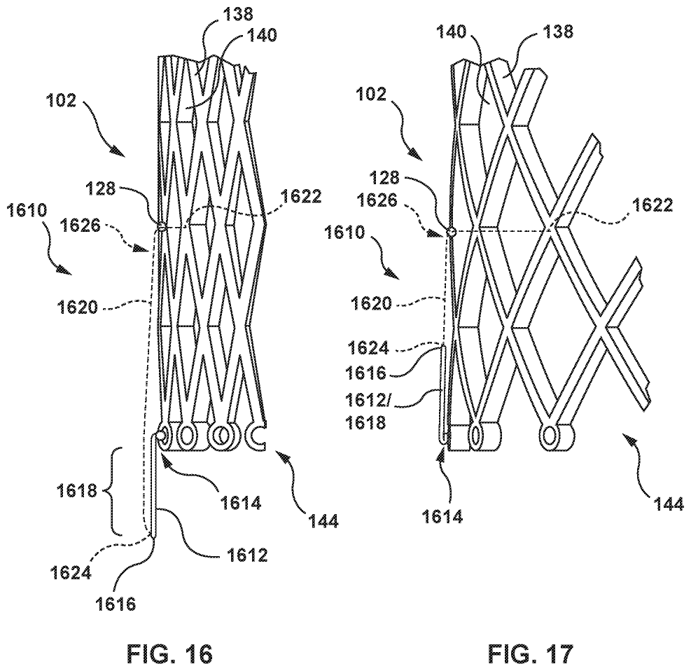

FIGS. 16 and 17 illustrate another embodiment of a sealing component 1610 in which the sealing component 1610 does not include any pleats in the delivery configuration. More particularly, FIG. 16 is a perspective view of a portion of a transcatheter valve prosthesis 1600 including the stent 102 in the compressed configuration and the sealing component 1610 shown in a delivery configuration while FIG. 17 is a perspective view of the portion of the transcatheter valve prosthesis 1600 with the stent 102 in the expanded configuration and the sealing component 1610 shown in a deployed configuration. The sealing component 1610 includes a cylindrical sleeve 1612 formed of a flexible material and a plurality of pulling sutures 1620 extending between the cylindrical sleeve 1612 and a stent 102. The plurality of pulling sutures 1620 are coupled to the stent 102 such that a body portion 1626 of each pulling suture 1620 can slide relative to the stent 102 when the stent 102 radially expands from the compressed configuration to the expanded configuration. The cylindrical sleeve 1612 is a cylindrical or component defining a central lumen or passageway and having a first circumferential edge 1614 and an opposing or second circumferential edge 1616. The first circumferential edge 1614 of the cylindrical sleeve 1612 is attached to the outer surface of the transcatheter valve prosthesis 1600 around the proximal end 144 of the stent 102. In this embodiment, the second circumferential edge 1616 of the cylindrical sleeve 1612 is not attached to the outer surface of the transcatheter valve prosthesis 1600 but rather is disposed proximal to the proximal end 144 of the stent 102 in the delivery configuration of the sealing component 1610.

In FIGS. 16 and 17, the pulling suture 1620 is shown in phantom to readily distinguish the pulling suture 1620 from the cylindrical sleeve 1612. The pulling suture 1620 is an elongated component having a first end 1622 and a second or opposing end 1624 with a body portion 1626 extending between the first and second ends 1622, 1624. The first end 1622 of the pulling suture 1620 is fixedly attached to the stent 102, the second end 1624 of the pulling suture 1620 is fixedly attached to the second circumferential edge 1616 of the cylindrical sleeve 1612, and the body portion 1626 of the pulling suture 1620 is coupled to the stent 102 such that the pulling suture 1620 can slide relative to the stent 102. As described above, the second end 1624 of the pulling suture 1620 is attached to the second circumferential edge 1616 of the cylindrical sleeve 1612.

Similar to the pulling suture 120, the pulling suture 1620 is configured to longitudinally pull, draw, haul, drag, move or otherwise reposition the intermediate portion 1618 of the cylindrical sleeve 1612 as the stent 102 radially expands from the compressed configuration to the expanded configuration. More particularly, the body portion 1626 of the pulling suture 1620A is slidingly disposed through a hole or opening 128 formed within a strut 138 of the stent 102. When the stent 102 radially expands, the second circumferential edge 1616 of the cylindrical sleeve 1612 inverts or rotates radially outward until the cylindrical sleeve 1612 lies flush or generally parallel to the outer surface 103 of the stent 102. In the deployed configuration of the sealing component 1610, the entire cylindrical sleeve 1610 is disposed distal of the proximal end 144 of the stent 102 and the cylindrical sleeve 1612 encircles the outer surface 103 of the stent 102. In this embodiment, the sealing component 1610 in the deployed configuration includes a single layer of the flexible material of the cylindrical sleeve 1612.

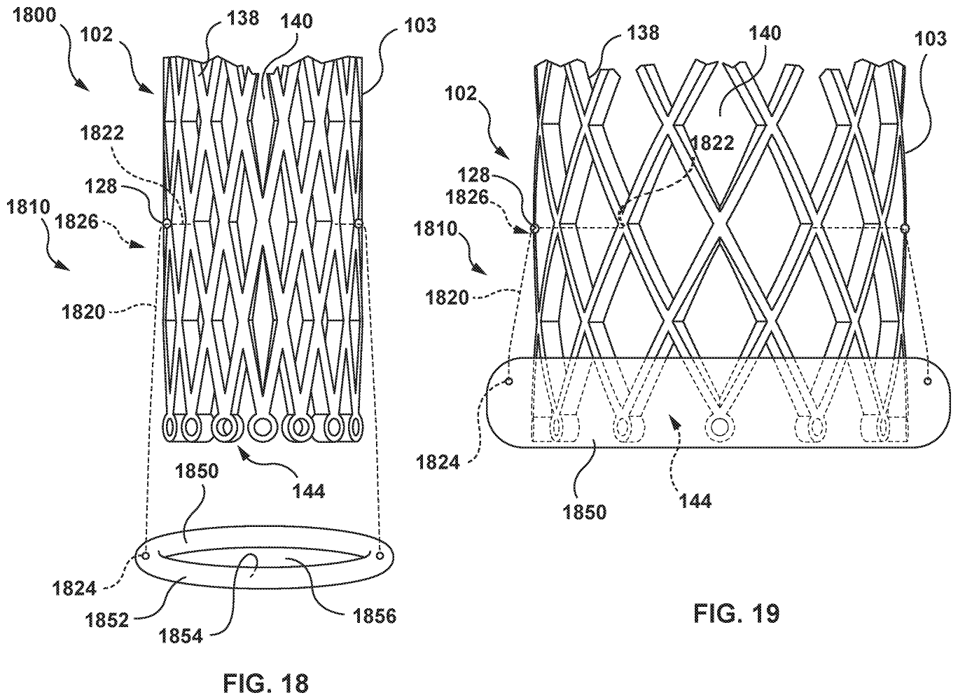

FIGS. 18 and 19 illustrate another embodiment of a sealing component 1810 in which the sealing component 1810 includes a donut-shaped component that is pulled into place via a plurality of pulling sutures 1820. More particularly, FIG. 18 is a perspective view of a transcatheter valve prosthesis 1800 including the stent 102 in the compressed configuration and the sealing component 1810 shown in a delivery configuration while FIG. 19 is a perspective view of the transcatheter valve prosthesis 1800 with the stent 102 in the expanded configuration and the sealing component 1810 shown in a deployed configuration. The sealing component 1810 includes a donut-shaped component 1850 formed of a flexible material and the plurality of pulling sutures 1820 extending between the donut-shaped component 1850 and the stent 102. The donut-shaped component 1850 is an annular component having an outer circumferential surface 1852 and an inner circumferential surface 1854 which defines a central lumen or passageway 1856 therethrough. The inner circumferential surface 1854 is dimensioned or configured to abut against the outer surface 103 of the stent 102 in the expanded configuration. Although it is shown in FIGS. 18-19 to have a length or height configurated to extend over the proximal or inflow portion of the stent 102, the length or height of the donut-shaped component 1850 may vary according to application and may extend over a greater or lesser amount of the stent 102. Suitable flexible materials for the donut-shaped component 1850 include but are not limited to a natural or biological material such as pericardium or another membranous tissue such as intestinal submucos or a low-porosity woven fabric, such as polyester, Dacron fabric, or PTFE. Porous materials advantageously provide a medium for tissue ingrowth. Further, the donut-shaped component 1850 may be pericardial tissue or may be a knit or woven polyester, such as a polyester or PTFE knit, both of which provide a medium for tissue ingrowth and have the ability to stretch to conform to a curved surface. Polyester velour fabrics may alternatively be used, such as when it is desired to provide a medium for tissue ingrowth on one side and a smooth surface on the other side. In the delivery configuration, as shown on FIG. 18, the donut-shaped component 1850 is spaced apart from and not coupled to the stent 102 except via the plurality of pulling sutures 1820. Further, the donut-shaped component 1850 is disposed proximal to the proximal end 144 of the stent 102 in the delivery configuration of the sealing component 1810.

The plurality of pulling sutures 1820 are coupled to the stent 102 such that a body portion 1826 of each pulling suture 1820 can slide relative to the stent 102 when the stent 102 radially expands from the compressed configuration to the expanded configuration. In FIGS. 18 and 19, the pulling suture 1820 is shown in phantom to readily distinguish the pulling suture 1820 from the donut-shaped component 1850. The pulling suture 1820 is an elongated component having a first end 1822 and a second or opposing end 1824 with the body portion 1826 extending between the first and second ends 1822, 1824. The first end 1822 of the pulling suture 1820 is fixedly attached to the stent 102, the second end 1824 of the pulling suture 1820 is fixedly attached to the donut-shaped component 1850, and the body portion 1826 of the pulling suture 1820 is coupled to the stent 102 such that the pulling suture 1820 can slide relative to the stent 102.

Similar to the pulling suture 120, the pulling suture 1820 is configured to longitudinally pull, draw, haul, drag, move or otherwise reposition the donut-shaped component 1850 as the stent 102 radially expands from the compressed configuration to the expanded configuration. More particularly, the body portion 1826 of the pulling suture 1820 is slidingly disposed through a hole or opening 128 formed within a strut 138 of the stent 102. When the stent 102 radially expands, the donut-shaped component 1850 is pulled distally towards the stent 102 until the donut-shaped component 1850 is disposed around the outer surface 103 of the stent 102. In the deployed configuration of the sealing component 1810, the donut-shaped component 1850 is disposed distal of the proximal end 144 of the stent 102 and the donut-shaped component 1850 encircles the outer surface 103 of the stent 102.

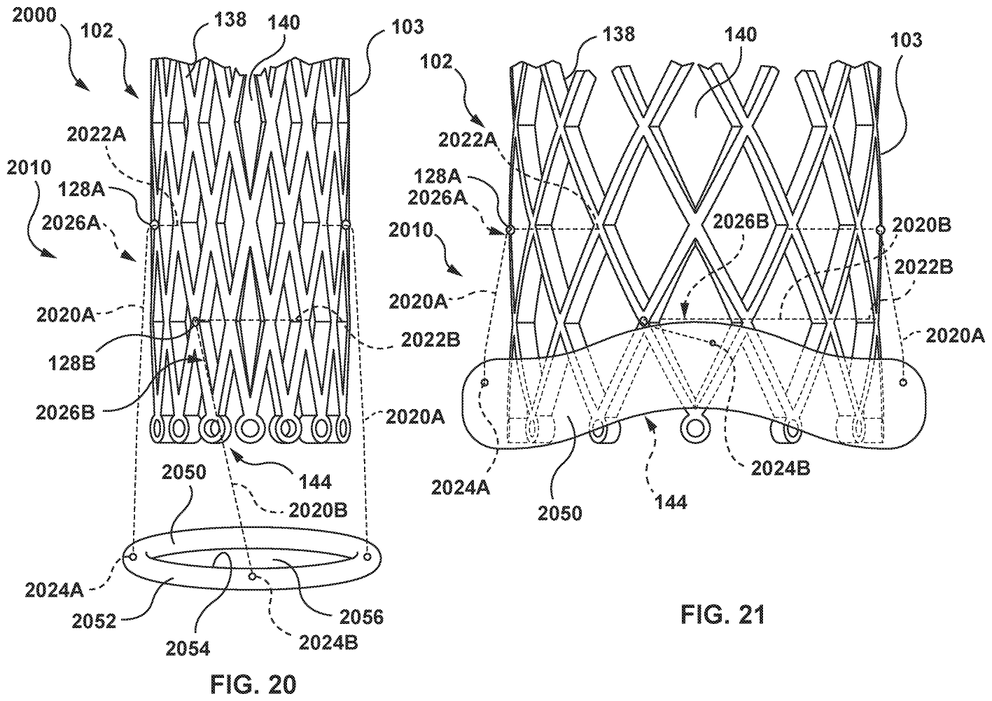

In the embodiment of FIGS. 18 and 19, each pulling suture 1820 has the same length and the donut-shaped component 1850 is pulled up or repositioned in a uniform manner such that the donut-shaped component 1850 in the deployed configuration forms a circular ring around the outer surface 103 of the stent 102. Stated another way, in the deployed configuration of the sealing component 1810, the donut-shaped component 1850 is a circular ring. FIGS. 20, 21, and 22 illustrate another embodiment of a sealing component 2010 in which the sealing component 2010 includes a donut-shaped component that is an undulating ring when the sealing component 2010 is in the deployed configuration. When the sealing component 2010 is in the deployed configuration, the material of the donut-shaped component bunches or gathers up into the undulating or wavy ring so that the bunched or gathered up material of the sealing component 2010 as deployed may further prevent and/or repair paravalvular leakage.

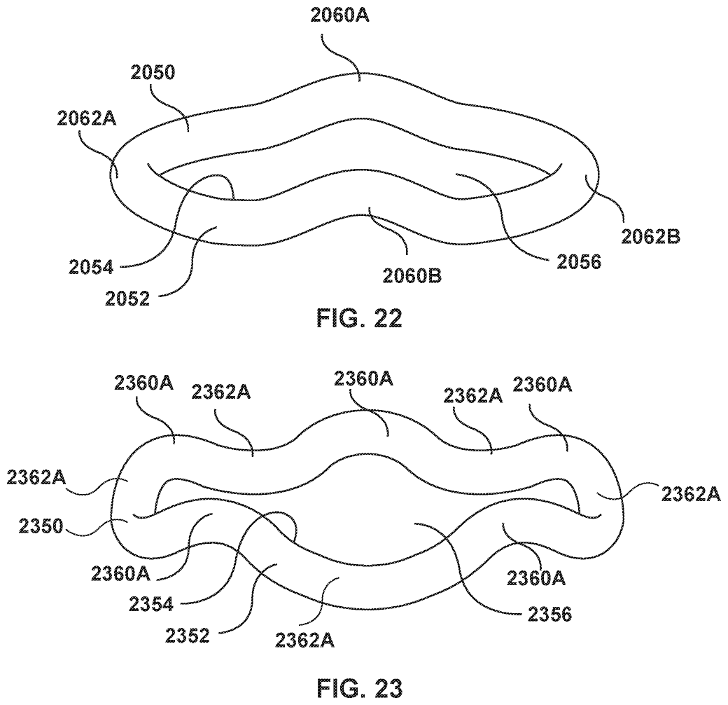

More particularly, FIG. 20 is a perspective view of a transcatheter valve prosthesis 2000 including the stent 102 in the compressed configuration and the sealing component 2010 shown in a delivery configuration while FIG. 21 is a perspective view of the transcatheter valve prosthesis 2000 with the stent 102 in the expanded configuration and the sealing component 2010 shown in a deployed configuration. The sealing component 2010 includes a donut-shaped component 2050 formed of a flexible material and a plurality of pulling sutures 2020A, 2020B extending between the donut-shaped component 2050 and the stent 102. FIG. 22 is a perspective view of the donut-shaped component 2050 in the deployed configuration, the donut-shaped component 2050 being removed from the transcatheter vale prosthesis 2000 for illustrative purposes only. The donut-shaped component 2050 is an annular component having an outer circumferential surface 2052 and an inner circumferential surface 2054 which defines a central lumen or passageway 2056 therethrough. The inner circumferential surface 2054 is dimensioned or configured to abut against the outer surface 103 of the stent 102 in the expanded configuration. Although it is shown in FIGS. 20-22 to have a length or height configurated to extend over the proximal or inflow portion of the stent 102, the length or height of the donut-shaped component 2050 may vary according to application and may extend over a greater or lesser amount of the stent 102. Suitable flexible materials for the donut-shaped component 2050 include but are not limited to a natural or biological material such as pericardium or another membranous tissue such as intestinal submucos or a low-porosity woven fabric, such as polyester, Dacron fabric, or PTFE. Porous materials advantageously provide a medium for tissue ingrowth. Further, the donut-shaped component 2050 may be pericardial tissue or may be a knit or woven polyester, such as a polyester or PTFE knit, both of which provide a medium for tissue ingrowth and have the ability to stretch to conform to a curved surface. Polyester velour fabrics may alternatively be used, such as when it is desired to provide a medium for tissue ingrowth on one side and a smooth surface on the other side. In the delivery configuration, as shown on FIG. 20, the donut-shaped component 2050 is spaced apart from and not coupled to the stent 102 except via the plurality of pulling sutures 2020A, 2020B. Further, the donut-shaped component 2050 is disposed proximal to the proximal end 144 of the stent 102 in the delivery configuration of the sealing component 2010.

In this embodiment, the sealing component 2010 includes a total of four pulling sutures including a first pair or set of pulling sutures 2020A coupled to opposing locations on the stent 102 and a second pair or set of pulling sutures 2020B coupled to opposing locations on the stent 102. One of the pulling sutures 2020B is not visible on FIGS. 20-21. The four pulling sutures 2020A, 2020B are equally circumferentially spaced around the stent 102 with each pulling suture 2020A being disposed between a pair of pulling sutures 2020B. Stated another way, the pair of pulling sutures 2020A are circumferentially offset by approximately ninety degrees from the pair of pulling sutures 2020B. As will be explained in more detail herein, the first set of pulling sutures 2020A is configured to longitudinally pull the donut-shaped component 2050 a greater amount than the second set of pulling sutures 2020B such that in the deployed configuration of the sealing component 2010, the donut-shaped component 2050 is an undulating ring.

The pulling sutures 2020A are coupled to the stent 102 such that a body portion 2026A of each pulling suture 2020A can slide relative to the stent 102 when the stent 102 radially expands from the compressed configuration to the expanded configuration. In FIGS. 20 and 21, the pulling suture 2020A is shown in phantom to readily distinguish the pulling suture 2020A from the donut-shaped component 2050. The pulling suture 2020A is an elongated component having a first end 2022A and a second or opposing end 2024A with the body portion 2026A extending between the first and second ends 2022A, 2024A. The first end 2022A of the pulling suture 2020A is fixedly attached to the stent 102, the second end 2024A of the pulling suture 2020A is fixedly attached to the donut-shaped component 2050, and the body portion 2026A of the pulling suture 2020A is coupled to the stent 102 such that the pulling suture 2020A can slide relative to the stent 102.

Similarly, the pulling sutures 2020B are coupled to the stent 102 such that a body portion 2026B of each pulling suture 2020B can slide relative to the stent 102 when the stent 102 radially expands from the compressed configuration to the expanded configuration. In FIGS. 20 and 21, the pulling suture 2020B is shown in phantom to readily distinguish the pulling suture 2020B from the donut-shaped component 2050. The pulling suture 2020B is an elongated component having a first end 2022B and a second or opposing end 2024B with the body portion 2026B extending between the first and second ends 2022B, 2024B. The first end 2022B of the pulling suture 2020B is fixedly attached to the stent 102, the second end 2024B of the pulling suture 2020B is fixedly attached to the donut-shaped component 2050, and the body portion 2026B of the pulling suture 2020B is coupled to the stent 102 such that the pulling suture 2020B can slide relative to the stent 102.

Each pulling suture 2020A, 2020B is configured to longitudinally pull, draw, haul, drag, move or otherwise reposition the donut-shaped component 2050 as the stent 102 radially expands from the compressed configuration to the expanded configuration. More particularly, the body portion 2026A of each pulling suture 2020A is slidingly disposed through a hole or opening 128A formed within a strut 138 of the stent 102 and the body portion 2026B of each pulling suture 2020B is slidingly disposed through a hole or opening 128B formed within a strut 138 of the stent 102. When the stent 102 radially expands, the donut-shaped component 2050 is pulled distally towards the stent 102 until the donut-shaped component 2050 is disposed around the outer surface 103 of the stent 102. In the deployed configuration of the sealing component 2010, the donut-shaped component 2050 is disposed distal of the proximal end 144 of the stent 102 and the donut-shaped component 2050 encircles the outer surface 103 of the stent 102.

In this embodiment, the pulling sutures 2020A, 2020B are configured to longitudinally pull, draw, haul, drag, move or otherwise reposition the donut-shaped component 2050 in a non-uniform manner such that the donut-shaped component 2050 in the deployed configuration forms a undulating or wavy ring around the outer surface 103 of the stent 102. More particularly, each suture of the first set of pulling sutures 2020A is longer than the each suture of the second set of pulling sutures 2020B. The relatively increased length of each pulling suture 2020A allows the pulling suture 2020A to span across a greater number of side openings 140 of the stent 102 such that the distance of travel of the donut-shaped component 2050 is relatively increased when the stent 102 radially expands. In addition, each suture of the first set of pulling sutures 2020A is coupled to the stent 102 closer to the proximal end 144 of the stent 102 than the each suture of the second set of pulling sutures 2020B. If the pulling sutures 2020A, 2020B were the same size and configured to travel the same distance when the stent 102 radially expands, different attachment locations of the pulling sutures 2020A, 2020B may vary the final longitudinal position of the donut-shaped component 2050 when the stent 102 radially expands. More particularly, if the pulling sutures 2020A, 2020B were the same size and configured to travel the same distance when the stent 102 radially expands, positioning each pulling suture 2020A closer to the proximal end 144 of the stent 102 allows the pulling suture 2020A to span across a greater number of side openings 140 of the stent 102 such that the distance of travel of the donut-shaped component 2050 is relatively increased when the stent 102 radially expands. Thus, the variable length of the pulling sutures 2020A, 2020B, as well as the variable attachment location relative to the proximal end 144 of the stent 102, may cause the pulling sutures 2020B to be configured to longitudinally pull or move the donut-shaped component 2050 a greater amount than the pulling sutures 2020A. As best shown on FIG. 22, opposing portions 2060A, 2060B of the donut-shaped component 2050 that are attached to the pulling sutures 2020B are pulled a greater amount than the opposing portions 2062A, 2062B of the donut-shaped component 2050 that are attached to the pulling sutures 2020A. As a result, the deployed configuration of the donut-shaped component 2050 includes bumps or bulges along the opposing portions 2060A, 2060B that are disposed closer to the distal end 142 of the stent 102 than the opposing portions 2062A, 2062B. Due to the bumps or bulges along the opposing portions 2060A, 2060B, the donut-shaped component 2050 in the deployed configuration form an undulating or wavy ring around the outer surface 103 of the stent 102.

As described above, in order to longitudinally pull or move the donut-shaped component 2050 a greater amount than the pulling sutures 2020A, the pulling sutures 2020B are longer than the pulling sutures 2020A and are also coupled to the stent 102 closer to the proximal end 144 of the stent 102 than the pulling sutures 2020A. However, in order to longitudinally pull or move the donut-shaped component 2050 a greater amount than the pulling sutures 2020A, the pulling sutures 2020B may be longer than the pulling sutures 2020A or may be coupled to the stent 102 closer to the proximal end 144 of the stent 102 than the pulling sutures 2020A. Stated another way, it is not required that the pulling sutures 2020A, 2020B have variable lengths in addition to variable attachment locations relative to the proximal end 144 of the stent 102. For example, in another embodiment, the pulling sutures 2020B are longer than the pulling sutures 2020A in order to longitudinally pull or move the donut-shaped component 2050 a greater amount than the pulling sutures 2020A but the pulling sutures 2020A, 2020B do not have a variable attachment location relative to the proximal end 144 of the stent 102. In another embodiment, the pulling sutures 2020B are coupled to the stent 102 closer to the proximal end 144 of the stent 102 than the pulling sutures 2020A in order to longitudinally pull or move the donut-shaped component 2050 a greater amount than the pulling sutures 2020A but the pulling sutures 2020A, 2020B do not have variable lengths.

Although the embodiment of FIGS. 20-22 is described with a total of four pulling sutures 2020A, 2020B and two resulting bumps or bulges along the opposing portions 2060A, 2060B, it will be understood by those of ordinary skill in the art that the sealing component 2010 may include more or fewer pulling sutures for repositioning the donut-shaped component 2050 and the number of resulting bumps or bulges of the donut-shaped component may vary from the embodiment of FIGS. 20-22. For example, FIG. 23 is a perspective view of a donut-shaped component 2350 in a deployed configuration according to another embodiment, with the donut-shaped component being removed from a transcatheter vale prosthesis for illustrative purposes only. Similar to the donut-shaped component 2050, the donut-shaped component 2350 is an annular component having an outer circumferential surface 2352 and an inner circumferential surface 2354 which defines a central lumen or passageway 2356 therethrough. The inner circumferential surface 2354 is dimensioned or configured to abut against the outer surface 103 of the stent 102 in the expanded configuration. The donut-shaped component 2350 in the deployed configuration includes a total of five portions 2360A that include bulges or bumps and a total of five portions 2362A that do not include bulges or bumps. Each portion 2360A of the donut-shaped component 2350 that includes a bulge or bump is coupled to a pulling suture that is configured to longitudinally pull or move the donut-shaped component 2350 a greater amount than the pulling sutures that are coupled to portions 2362A that do not include bulges or bumps. In an embodiment, the donut-shaped component is configured to include at two portions that include bulges or bumps and may include between two and six portions that include bulges or bumps.

According to a first embodiment hereof, a transcatheter valve prosthesis includes a stent, a prosthetic valve component disposed within and secured to the stent, and a sealing component. The stent has a compressed configuration for delivery within a vasculature and an expanded configuration for deployment within a native heart valve. The sealing component includes a donut-shaped component and at least one pulling suture extending between the donut-shaped component and the stent. The at least one pulling suture is coupled to the stent such that the pulling suture can slide relative to the stent when the stent radially expands from the compressed configuration to the expanded configuration. The sealing component has a delivery configuration in which the donut-shaped component is disposed proximal of a proximal end of the stent and a deployed configuration in which the donut-shaped component is disposed distal of the proximal end of the stent. The pulling suture is configured to longitudinally pull the donut-shaped component as the stent radially expands from the compressed configuration to the expanded configuration.

In an aspect of the first embodiment, and in combination with any other aspects herein, in the deployed configuration of the sealing component the donut-shaped component encircles an outer surface of the stent.

In an aspect of the first embodiment, and in combination with any other aspects herein, in the deployed configuration of the sealing component the donut-shaped component is a circular ring.

In an aspect of the first embodiment, and in combination with any other aspects herein, in the deployed configuration of the sealing component the donut-shaped component is an undulating ring.

In an aspect of the first embodiment, and in combination with any other aspects herein, the sealing component includes a plurality of pulling sutures extending between the donut-shaped component and the stent. The pulling sutures are circumferentially spaced around the stent.

In an aspect of the first embodiment, and in combination with any other aspects herein, the plurality of pulling sutures includes a first set of pulling sutures and a second set of pulling sutures. The first set of pulling sutures is configured to longitudinally pull the donut-shaped component a greater amount than the second set of pulling sutures such that in the deployed configuration of the sealing component the donut-shaped component is an undulating ring.

In an aspect of the first embodiment, and in combination with any other aspects herein, each suture of the first set of pulling sutures is longer than the each suture of the second set of pulling sutures.

In an aspect of the first embodiment, and in combination with any other aspects herein, each suture of the first set of pulling sutures is coupled to the stent closer to the proximal end of the stent than the each suture of the second set of pulling sutures.

In an aspect of the first embodiment, and in combination with any other aspects herein, the donut-shaped component is an annular component having an outer circumferential surface and an inner circumferential surface which defines a central lumen therethrough. The inner circumferential surface is configured to abut against the outer surface of the stent in the deployed configuration.

In an aspect of the first embodiment, and in combination with any other aspects herein, in the deployed configuration the donut-shaped component is spaced apart from and not coupled to the stent except via the at least one pulling suture.

In an aspect of the first embodiment, and in combination with any other aspects herein, radial expansion of the stent from the compressed configuration to the expanded configuration causes the pulling suture to longitudinally reposition the donut-shaped component such that the sealing component has a delivery configuration in which the donut-shaped component is disposed at a first longitudinal position and the sealing component has a deployed configuration in which the donut-shaped component is disposed at a second longitudinal position. The first longitudinal portion is proximal of a proximal end of the stent and the second longitudinal portion is distal of the proximal end of the stent.

In an aspect of the first embodiment, and in combination with any other aspects herein, a first end of the at least one pulling suture is attached to the stent, a second end of the at least one pulling suture is attached to the donut-shaped component, and a body portion of the at least one pulling suture is coupled to the stent such that the pulling suture can slide relative to the stent when the stent radially expands from the compressed configuration to the expanded configuration.

According to a second embodiment hereof, a transcatheter valve prosthesis includes a stent, a prosthetic valve component disposed within and secured to the stent, a sealing component, and at least one pulling suture extending between the sealing component and the stent. The stent has a compressed configuration for delivery within a vasculature and an expanded configuration for deployment within a native heart valve. A first end of the at least one pulling suture is attached to the stent, a second end of the at least one pulling suture is attached to the sealing component, and a body portion of the at least one pulling suture is coupled to the stent such that the pulling suture can slide relative to the stent when the stent radially expands from the compressed configuration to the expanded configuration. The sealing component has a delivery configuration in which the sealing component is disposed proximal of a proximal end of the stent and is longitudinally spaced apart from and not coupled to the stent except via the at least one pulling suture. The sealing component has a deployed configuration in which the sealing component is disposed distal of the proximal end of the stent and encircles an outer surface of the stent. The pulling suture is configured to longitudinally reposition the sealing component as the stent radially expands from the compressed configuration to the expanded configuration.

In an aspect of the second embodiment, and in combination with any other aspects herein, in the deployed configuration of the sealing component the donut-shaped component is a circular ring.

In an aspect of the second embodiment, and in combination with any other aspects herein, in the deployed configuration of the sealing component the donut-shaped component is an undulating ring.

In an aspect of the second embodiment, and in combination with any other aspects herein, a plurality of pulling sutures extend between the sealing component and the stent. The pulling sutures are circumferentially spaced around the stent.

In an aspect of the second embodiment, and in combination with any other aspects herein, the plurality of pulling sutures includes a first set of pulling sutures and a second set of pulling sutures. The first set of pulling sutures is configured to longitudinally pull the donut-shaped component a greater amount than the second set of pulling sutures such that in the deployed configuration of the sealing component the donut-shaped component is an undulating ring.

In an aspect of the second embodiment, and in combination with any other aspects herein, each suture of the first set of pulling sutures is longer than the each suture of the second set of pulling sutures.

In an aspect of the second embodiment, and in combination with any other aspects herein, each suture of the first set of pulling sutures is coupled to the stent closer to the proximal end of the stent than the each suture of the second set of pulling sutures.

In an aspect of the second embodiment, and in combination with any other aspects herein, the sealing component is an annular component having an outer circumferential surface and an inner circumferential surface which defines a central lumen therethrough. The inner circumferential surface is configured to abut against the outer surface of the stent in the deployed configuration.

While various embodiments according to the present invention have been described above, it should be understood that they have been presented by way of illustration and example only, and not limitation. For example, although the sealing component 110 is shown in the above embodiments as encircling the proximal end 144 of the transcatheter valve prosthesis 100, it will be understood by one of ordinary skill in the art that the sealing component 110 may alternatively be disposed at other longitudinal positions along the transcatheter valve prosthesis. For example, the sealing component may be disposed around the distal end 142 of the transcatheter valve prosthesis 100, or only around a middle or intermediate waist portion of the transcatheter valve prosthesis. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A transcatheter valve prosthesis comprising:
a stent having a compressed configuration for delivery within a vasculature and an expanded configuration for deployment within a native heart valve;
a prosthetic valve component disposed within and secured to the stent; and
a sealing component including a donut-shaped component and at least one pulling suture extending between the donut-shaped component and the stent, the at least one pulling suture being coupled to the stent such that the pulling suture can slide relative to the stent when the stent radially expands from the compressed configuration to the expanded configuration,
wherein the sealing component has a delivery configuration in which the donut-shaped component is disposed proximal of a proximal end of the stent and a deployed configuration in which the donut-shaped component is disposed distal of the proximal end of the stent, the pulling suture being configured to longitudinally pull the donut-shaped component as the stent radially expands from the compressed configuration to the expanded configuration.

2. The transcatheter valve prosthesis of claim 1, wherein in the deployed configuration of the sealing component the donut-shaped component encircles an outer surface of the stent.

3. The transcatheter valve prosthesis of claim 1, wherein in the deployed configuration of the sealing component the donut-shaped component is a circular ring.

4. The transcatheter valve prosthesis of claim 1, wherein in the deployed configuration of the sealing component the donut-shaped component is an undulating ring.

5. The transcatheter valve prosthesis of claim 1, wherein the sealing component includes a plurality of pulling sutures extending between the donut-shaped component and the stent, the pulling sutures being circumferentially spaced around the stent.

6. The transcatheter valve prosthesis of claim 5, wherein the plurality of pulling sutures includes a first set of pulling sutures and a second set of pulling sutures, the first set of pulling sutures being configured to longitudinally pull the donut-shaped component a greater amount than the second set of pulling sutures such that in the deployed configuration of the sealing component the donut-shaped component is an undulating ring.

7. The transcatheter valve prosthesis of claim 6, wherein each suture of the first set of pulling sutures is longer than the each suture of the second set of pulling sutures.

8. The transcatheter valve prosthesis of claim 6, wherein each suture of the first set of pulling sutures is coupled to the stent closer to the proximal end of the stent than the each suture of the second set of pulling sutures.

9. The transcatheter valve prosthesis of claim 1, wherein the donut-shaped component is an annular component having an outer circumferential surface and an inner circumferential surface which defines a central lumen therethrough, the inner circumferential surface being configured to abut against the outer surface of the stent in the deployed configuration.

10. The transcatheter valve prosthesis of claim 1, wherein in the deployed configuration the donut-shaped component is spaced apart from and not coupled to the stent except via the at least one pulling suture.

11. The transcatheter valve prosthesis of claim 10, wherein radial expansion of the stent from the compressed configuration to the expanded configuration causes the pulling suture to longitudinally reposition the donut-shaped component such that the sealing component has a delivery configuration in which the donut-shaped component is disposed at a first longitudinal position and the sealing component has a deployed configuration in which the donut-shaped component is disposed at a second longitudinal position, wherein the first longitudinal portion is proximal of a proximal end of the stent and wherein the second longitudinal portion is distal of the proximal end of the stent.

12. The transcatheter valve prosthesis of claim 1, wherein a first end of the at least one pulling suture is attached to the stent, a second end of the at least one pulling suture is attached to the donut-shaped component, and a body portion of the at least one pulling suture is coupled to the stent such that the pulling suture can slide relative to the stent when the stent radially expands from the compressed configuration to the expanded configuration.

13. A transcatheter valve prosthesis comprising:
a stent having a compressed configuration for delivery within a vasculature and an expanded configuration for deployment within a native heart valve;
a prosthetic valve component disposed within and secured to the stent;
a sealing component; and
at least one pulling suture extending between the sealing component and the stent, wherein a first end of the at least one pulling suture is attached to the stent, a second end of the at least one pulling suture is attached to the sealing component, and a body portion of the at least one pulling suture is coupled to the stent such that the pulling suture can slide relative to the stent when the stent radially expands from the compressed configuration to the expanded configuration, and
wherein the sealing component has a delivery configuration in which the sealing component is disposed proximal of a proximal end of the stent and is longitudinally spaced apart from and not coupled to the stent except via the at least one pulling suture, and
wherein the sealing component has a deployed configuration in which the sealing component is disposed distal of the proximal end of the stent and encircles an outer surface of the stent, the pulling suture being configured to longitudinally reposition the sealing component as the stent radially expands from the compressed configuration to the expanded configuration.

14. The transcatheter valve prosthesis of claim 13, wherein in the deployed configuration of the sealing component the donut-shaped component is a circular ring.

15. The transcatheter valve prosthesis of claim 13, wherein in the deployed configuration of the sealing component the donut-shaped component is an undulating ring.

16. The transcatheter valve prosthesis of claim 13, wherein a plurality of pulling sutures extending between the sealing component and the stent, the pulling sutures being circumferentially spaced around the stent.

17. The transcatheter valve prosthesis of claim 16, wherein the plurality of pulling sutures includes a first set of pulling sutures and a second set of pulling sutures, the first set of pulling sutures being configured to longitudinally pull the donut-shaped component a greater amount than the second set of pulling sutures such that in the deployed configuration of the sealing component the donut-shaped component is an undulating ring.

18. The transcatheter valve prosthesis of claim 17, wherein each suture of the first set of pulling sutures is longer than the each suture of the second set of pulling sutures.

19. The transcatheter valve prosthesis of claim 17, wherein each suture of the first set of pulling sutures is coupled to the stent closer to the proximal end of the stent than the each suture of the second set of pulling sutures.

20. The transcatheter valve prosthesis of claim 13, wherein the sealing component is an annular component having an outer circumferential surface and an inner circumferential surface which defines a central lumen therethrough, the inner circumferential surface being configured to abut against the outer surface of the stent in the deployed configuration.
